(19) United States
(12) Plant Patent Application Publication
(10) Pub. No.: US 2005/0216999 P1
(43) Pub. Date: Sep. 29, 2005

(54) PETUNIA PLANT NAMED 'SUNPATIKI'

(50) PETUNIA HYBRIDA; PETUNIA

(76) Inventors: Kazunari Iwaki, Omihachiman (JP); Takuro Ishihara, Higashikurume (JP); Shinya Miyano, Katori-gun (JP)

Correspondence Address: CHRISTIE, PARKER & HALE, LLP, PO BOX 7068, PASADENA, CA, 91109-7068, US

(21) Appl. No.: 10/811,241

(22) Filed: Mar. 25, 2004

(51) Int. Cl. A01H 5/00

(52) U.S. Cl. PLT/356

(57) ABSTRACT

Disclosed herein is a new and distinct variety of Petunia plant having a decumbent habit and short stems. The Petunia plant has abundant branching, and a great profusion of blooms, the whole plant remaining in bloom for a considerable period of time. The flowers are single and small, the petals having pale yellow-green with brilliant yellow-green and brilliant greenish-yellow veins. The inside color of the corolla throat is brilliant greenish-yellow and the outside of the corolla tube is light yellow-green. The plant exhibits high resistance to rain, heat, cold and disease.

### BOTANICAL/COMMERCIAL CLASSIFICATION

Petunia hybrida/Petunia Plant

### VARIETAL DENOMINATION

cv. ‘Sunpatiki’

### BACKGROUND OF THE VARIETY

The present invention relates to a new and distinct variety of Petunia plant, which originated from the crossing of a Petunia hybrid variety called ‘152-3’ (unpatented) as the female parent and a seedling called ‘Summer Sun’ (unpatented) as the male parent.

The Petunia is a very popular plant that is used for flower bedding and potting in the summer season. There are only a few Petunia varieties that do not have an upright growth habit and that have a high resistance to rain, heat, and disease. Petunias of the ‘Revolution’ series include ‘Revolution Purple pink’ (U.S. Plant Pat. No. 6,915), ‘Revolution Brilliant pink’ (U.S. Plant Pat. No. 6,914), ‘Revolution Brilliantpink-Mini’ (U.S. Plant Pat. No. 6,899), and ‘Revolution Blue vein’ (U.S. Plant Pat. No. 9,322). These are decumbent type plants having long stems, a lower plant height, abundant branching, and a high resistance to heat, rain and disease. Likewise, there are only a few Petunia varieties having a great profusion of small size flowers, yellow colored flower petals and a high resistance to rain, heat, and diseases. Accordingly, this invention was aimed at obtaining a new Petunia variety having yellow colored petals, together with the above features.

### Progress

The female parent ‘152-3’ used in the crossing of ‘Sunpatiki’ is a strain of our breeding lines, having a spreading growth habit with many branches. It has very small single flowers, the petals having yellowish white color.

The male parent ‘Summer Sun’ used in the crossing of ‘Sunpatiki’ is a cultivar having an erect growth habit with very few branches, and thick stems. It has large vivid yellow flowers. The seed of ‘Summer Sun’ is commercially available.

In January 2000, crossing of ‘152-3’ as the female parent and ‘Summer Sun’ as the pollen parent was conducted at Yokaichi-shi, Shiga-ken, Japan. In May 2000, 60 seedlings were obtained from that crossing. These seedlings were grown in pots in glasshouses and were evaluated. One seedling was selected in view of its growth habit, flower size and color in September 2000. That seedling was propagated by cutting and a trial was carried out by flower potting and bedding from April to September 2001 at Yokaichi-shi, Shiga-ken, Japan. The botanical characteristics of that plant were then examined, using similar varieties ‘Sunpatire’ (U.S. Ser. No. 10/611,359) and ‘Fantasy Crystal Red’ (unpatented) for comparison. As a result, it was concluded that this Petunia plant is distinguishable from any other variety whose existence is known to us, and is uniform and stable in its characteristics. The new variety of Petunia plant was named ‘Sunpatiki’.

In the following description, the color-coding is in accordance with the Horticultural Colour Chart of The Royal Horticultural Society, London, England (R.H.S. Colour Chart).

### SUMMARY OF THE VARIETY

This new variety is unlike any Petunia commercially available as evidenced by the following unique combinations of characteristics.

1. Spreading growth habit with short stems.

2. Abundant branching and a great profusion of blooms.

3. The flowers are single and small. The petal color is pale yellow green (near R.H.S.1D) with brilliant yellow green (near R.H.S.151C) and brilliant greenish-yellow (near R.H.S. 7B) venation.

4. The plant has a high resistance to rain, cold, heat and disease.

The new variety ‘Sunpatiki’ differs from the similar variety ‘Sunpatire’ in the following points.

1. The leaf shape of ‘Sunpatiki’ is elliptic, while that of ‘Sunpatire’ is lanceolate.

2. The leaf of ‘Sunpatiki’ is smaller and thinner than that of ‘Sunpatire’.

3. The petal color of ‘Sunpatiki’ is pale yellow green (near R.H.S.1D) with brilliant yellow green (near R.H.S.151C) and brilliant greenish yellow (near R.H.S.7B) venation, while that of ‘Sunpatire’ is vivid red (near R.H.S.N57A).

4. The apex shape of petal of ‘Sunpatiki’ is truncate while that of ‘Sunpatire’ is rounded.

The new variety ‘Sunpatiki’ differs from the similar variety ‘Fantasy Crystal Red’ in the following points.

1. The growth habit of ‘Sunpatiki’ is spreading, while that of ‘Fantasy Crystal Red’ is erect.

2. The stem length of ‘Sunpatiki’ is shorter than that of ‘Fantasy Crystal Red’.

3. The leaf of ‘Sunpatiki’ is thinner than that of ‘Fantasy Crystal Red’.

4. The petal color of ‘Sunpatiki’ is pale yellow green (near R.H.S.1D) with brilliant yellow green (near R.H.S.151C) and brilliant greenish yellow (near R.H.S. 7B) venation, while that of ‘Fantasy Crystal Red’ is vivid red (R.H.S.52A) with strong red (R.H.S.53C) venation.

5. The apex shape of petal of ‘Sunpatiki’ is truncate, while that of ‘Fantasy Crystal Red’ is obtuse.

6. The heat resistance of ‘Sunpatiki’ is stronger than that of ‘Fantasy Crystal Red’.

The new variety of Petunia plant ‘Sunpatiki’ was asexually reproduced by use of cutting at Yokaichi-shi, Shiga-ken, Japan, and the homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during July 2003 while cultivating under the trial field in 15 cm pots at an age of approximately 6 months at Yokaichi-shi, Shiga-ken, Japan.

FIG. 1 is a photograph of a typical plant of the new variety of Petunia plant ‘Sunpatiki’ while growing in a pot.

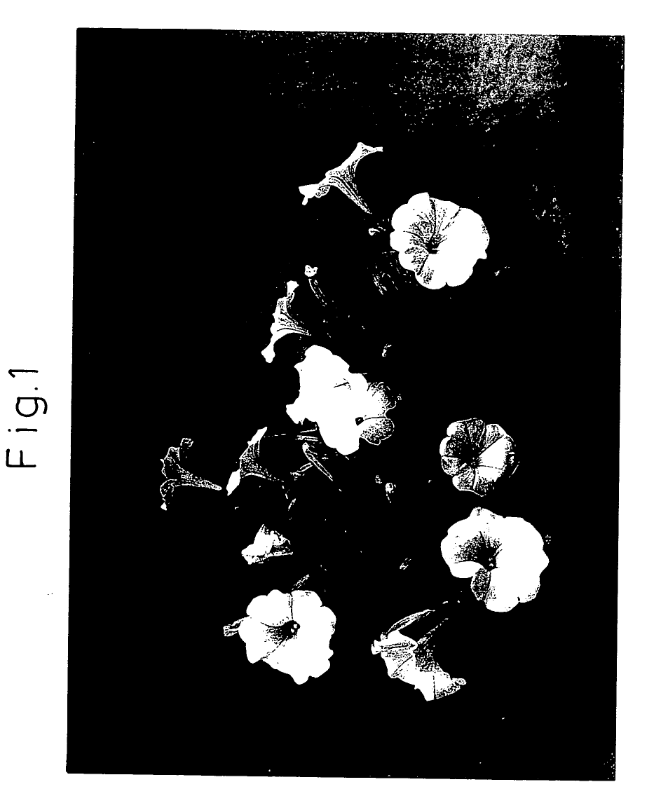

FIG. 2 is a close view of flowers and leaves of the new variety of Petunia plant ‘Sunpatiki’.

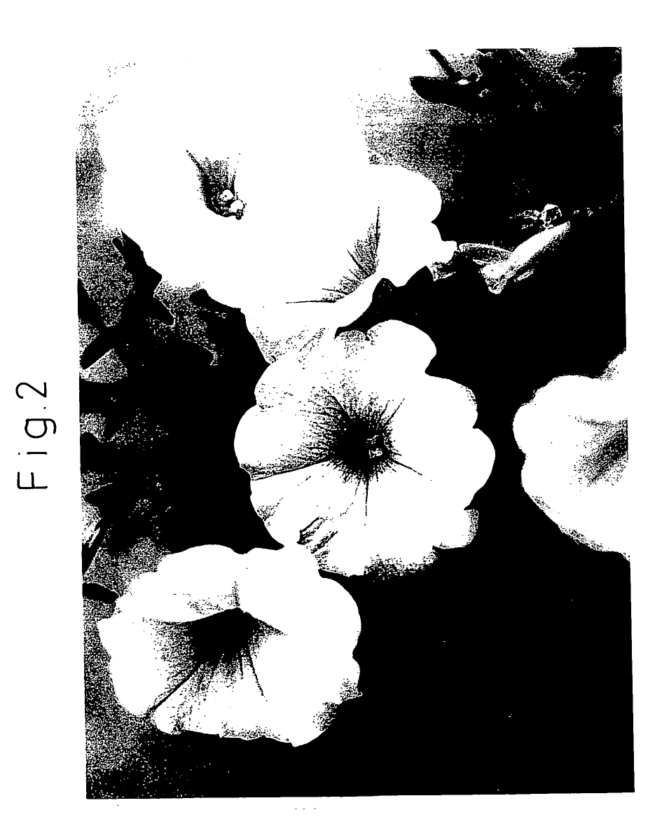

### DESCRIPTION OF THE VARIETY

The botanical characteristics of the new and distinct variety of Petunia plant named ‘Sunpatiki’ are as follows when observed during July at Yokaichi-shi, Shiga-ken, Japan at an age of approximately 6 months.
Plant:
   Growth habit.—Spreading.
   Plant height.—Approximately 19 cm.
   Spreading area of plant.—Approximately 14 cm.
   Blooming period.—Early April to late October in the southern Kanto area, Japan. The plant shape does not change throughout this period. A typical flower commonly lasts approximately 5 days on the plant when experiencing a temperature of approximately 20° C.
Stem:
   Length.—Approximately 1.9 cm.
   Thickness.—Approximately 2.0 mm.
   Pubescence.—Normal.
   Branching.—Abundant.
   Internode length.—Approximately 1.3 cm.
   Color.—Near R.H.S. 144B (vivid yellow-green).
Leaf:
   Whole shape.—Elliptic with entire margin. The apex shape is acute, and the base shape is attenuate.
   Length.—Approximately 2.7 cm.
   Width.—Approximately 1.7 cm.
   Color.—Upper side color is near R.H.S. 137A (moderate olive-green). Lower side color is near R.H.S. 146A (moderate yellow-green).
   Thickness.—Approximately 0.2 mm.
   Pubescence.—Sparse.
Buds:
   Shape.—Cylindrical.
   Length.—Approximately 4.5 cm.
   Diameter.—Approximately 1.0 cm.
   Color.—Near R.H.S. 1C with venation near R.H.S. 145B.
Flower:
   Depth.—approximately 5.0 cm.
   Tube length.—Approximately 2.6 cm.
   Throat diameter.—Distal end: approximately 1.1 cm.
   Tube diameter.—Proximal end: approximately 4.0 mm.
   Facing direction.—Slanted upward.
   Type.—Single.
   Shape.—Funnel-shaped, with five-fissures.
   Shape of petal tip.—Truncate.
   Lobation.—Shallow
   Waviness of petal.—Weak.
   Diameter.—Approximately 4.4 cm.
   Color.—Petal; near R.H.S. 1D (pale yellow-green) with venation near R.H.S.151C (brilliant yellow green) and near R.H.S.7B (brilliant greenish-yellow). Inside color of the corolla throat, near R.H.S. 7B (brilliant greenish-yellow). Outside color of the corolla tube; near R.H.S. 1C (light yellow-green).
Petals:
   Width.—Approximately 3.0 cm.
   Length from throat.—Approximately 2.5 cm.
   Shape.—Very broadly obovate.
   Margin.—Entire.
   Texture. Smooth.
   Color.—Lower surface, near R.H.S. 11C with near R.H.S. 11A venation.
   Reproductive organs.—1 normal pistil and 5 normal stamens. Color of pistil is near R.H.S. 145D (moderate yellow-green). Color of stamen is near R.H.S. 10D (pale yellowish-green).
Peduncle:
   Diameter.—Approximately 0.9 mm.
   Length.—Approximately 1.6 cm.
   Color.—Near R.H.S. 144A.
   Surface.—Pubescent.
   Calyx.—Narrow. 5 sepals fused at the base.
Sepals:
   Shape.—Narrow elliptic.
   Apex shape.—Rounded.
   Base.—Fused.
   Margin.—Entire.
   Surface.—Pubescent.
   Length.—Approximately 1.4 cm.
   Width.—Approximately 4.0 mm.
   Color.—Upper surface, near R.H.S. 137C, lower surface near R.H.S. 137C.
   Physiological and ecological characteristics.—High resistance to rain, cold, heat and disease. Moderate resistance to pests.

This new variety of Petunia plant is most suitable for flower bedding and potting, particularly in hanging pots or planters. Pinching of old blossoms will enhance the formation of new blossoms.

1. A new and distinct variety of Petunia plant named ‘Sunpatiki’, substantially as herein illustrated and described.
